(19) United States
(12) Patent Application Publication
(10) Pub. No.: US 2021/0137722 A1
(43) Pub. Date: May 13, 2021

(54) ASSIST DEVICE

(71) Applicant: JTEKT CORPORATION, Osaka-shi (JP)

(72) Inventors: Tomoki ARAI, Kitakatsuragi-gun (JP); Hiromichi OHTA, Kariya-shi (JP); Kazuyoshi OHTSUBO, Chiryu-shi (JP); Yoshitaka YOSHIMI, Kashiba-shi (JP)

(73) Assignee: JTEKT CORPORATION, Osaka-shi (JP)

(21) Appl. No.: 17/097,772

(22) Filed: Nov. 13, 2020

(30) Foreign Application Priority Data
Nov. 13, 2019 (JP) 2019-205688
Nov. 21, 2019 (JP) 2019-210358

(51) Int. Cl. A61F 5/02; A61F 5/01

(52) U.S. Cl. CPC A61F 5/028; A61F 2005/0155; A61F 5/0102

(57) ABSTRACT

An assist device includes a first harness, a second harness, and a grip portion. The first harness is configured to be fitted to at least one of a shoulder region and a breast region of a user. The second harness is configured to be fitted to a leg region or a waist region of the user. The grip portion is to be held by a person other than the user, the grip portion being attached to at least one of the first harness and the second harness.

### CROSS-REFERENCE TO RELATED APPLICATION

This application claims priority to Japanese Patent Application No. 2019-210358 filed on Nov. 21, 2019 and Japanese Patent Application No. 2019-205688 filed on Nov. 13, 2019, each incorporated herein by reference in its entirety.

### BACKGROUND

### 1. Technical Field

The present disclosure of the present disclosure relates to an assist device.

### 2. Description of Related Art

Various assist devices that assist a user's work by being worn on the body of the user (human being) have been proposed. For example, even where a user lifts a heavy object, an assist device enables the user to perform the work with a small strength (burden). Such assist device is disclosed in, for example, Japanese Unexamined Patent Application Publication No. 2018-199205 (JP 2018-199205 A).

### SUMMARY

The assist device disclosed in JP 2018-199205 A includes a frame made of, e.g., a metal, the frame being fitted to a user. An output of an actuator mounted in the frame is transmitted to the upper body and the lower body of the user through a link mechanism. Consequently, for example, a motion of lifting a heavy object is assisted.

Examples of motions for which a user needs assistance include motions of assisting (helping) a person such as a patient or an elderly person (hereinafter also referred to as “care-receiver”) in activities of daily living in addition to motions involving a large burden such as lifting a heavy object. When a user performs work involving a heavy burden, a high-power assist device such as one disclosed in JP 2018-199205 A is effective.

However, when a user helps (assists) a care-receiver, a high-power assist device may be excessive in performance. Also, a high-power assist device uses many rigid members such as a link mechanism and a frame made of, e.g., metal, and has a solid configuration in order to achieve a high output. Therefore, the assist device is heavy in weight and movement of the user is restricted by the rigid members.

Therefore, the inventor of the present disclosure has already proposed assist devices that are light in weight and provide good wear comfort (for example, Japanese Patent Application No. 2019-043462). Each of such assist devices includes a first harness to be fitted to a shoulder region of a user, second harnesses to be fitted to left and right leg regions of the user, a belt body provided so as to extend to the first harness and to the second harnesses along the back side of the user, and an actuator. The actuator is provided in the first harness and enables winding and unwinding a part of the belt body.

Such assist devices as above may be used in, for example, hospitals or nursing care facilities. In such case, a user sometimes gives assistance in a posture in which the user is in physical contact with a care-receiver, and thus, operation of an assist device worn by the user may be hindered by the assist device coming into contact with the care-receiver. Also, a user (caregiver) wearing an assist device helps a care-receiver's posture change by, e.g., raising the care-receiver in his/her arms. At that time, while the caregiver holds the care-receiver in his/her arms, the care-receiver sometimes tries to seize the caregiver. If the care-receiver seizes a movable portion, for example, a belt body, of the assist device, the assist device may fail to properly operate.

Therefore, the present disclosure provides an assist device that provides good wear comfort and can operate smoothly when a user gives assistance. Also, the present disclosure provides an assist device that properly operates when a user wearing the assist device performs work of, for example, changing another person's posture.

An assist device according to a first aspect of the present disclosure includes: a first harness configured to be fitted to at least one of a shoulder region and a breast region of a user; a second harness configured to be fitted to a leg region or a waist region of the user; a belt body provided so as to extend to the first harness and to the second harness along the back side of the user; an actuator provided in the first harness or the second harness and configured to wind and unwind a part of the belt body; and a grip portion to be held by a person other than the user, the grip portion being attached to at least one of the first harness and the second harness.

With the assist device according to the first aspect of the present disclosure, the belt body is provided so as to extend to the first harness and to the second harness along the back side of a user. By the actuator winding the belt body, tension acts on the belt body. The tension causes generation of an assist force for assisting the user's work, which reduces a burden on the body of the user. For example, in, e.g., a hospital or a nursing care facility, when a caregiver who is a user of the assist device changes his/her posture from a forward-tilted posture to an upright posture while holding a care-receiver who is a person other than the user in his/her arms in order to raise the care-receiver, tension acts on the belt body by the actuator winding the belt body. The tension makes it easy for the caregiver to change his/her posture from a forward-tilted posture to an upright posture and thus reduces a burden on the body of the caregiver.

Then, for example, when the caregiver changes his/her posture from a forward-tilted posture to an upright posture while holding the care-receiver in his/her arms, the care-receiver tries to seize the caregiver. In this case, letting the care-receiver hold the grip portion enables preventing the belt body from being held and operation of the belt body is thus not hindered. Therefore, the assist device properly operates.

In the assist device according to the first aspect of the present disclosure, the first harness may include a back body portion that faces the back of the user and a shoulder belt for fixing the first harness to the user, the shoulder belt connecting with the back body portion, and the grip portion may be attached to the back body portion. With the assist device according to the first aspect of the present disclosure, the grip portion is on the rear side of the first harness, that is, on the back side of the caregiver. Therefore, where the care-receiver flings his/her arms around the upper body of the caregiver, the care-receiver can easily hold the grip portion.

In the assist device according to the first aspect of the present disclosure, the grip portion may form a loop shape jointly with the back body portion by the opposite end sides of the grip portion being connected respectively to an upper portion and a lower portion of each of a left portion and a right portion of the back body portion. With the assist device according to the first aspect of the present disclosure, the grip portion has a loop shape and thus the care-receiver can easily hold the grip portion. Alternatively, the care-receiver can push his/her body against the caregiver by putting his/her arms through the grip portion having a loop shape.

In the assist device according to the first aspect of the present disclosure, the actuator may be mounted on a base attached to the back body portion, and the grip portion may be attached to the back body portion. With the assist device according to the first aspect of the present disclosure, a force acting on the grip portion as a result of the care-receiver holding the grip portion is transmitted to the first harness through the back body portion. On the other hand, the forces acting on the grip portion is less likely to act on the actuator, and thus, operation such as winding of the belt body by the actuator is not hindered by the force.

With the assist device according to the first aspect of the present disclosure, operation of the belt body is not hindered and the assist device properly operates.

An assist device according to a second aspect of the present disclosure includes: a first harness configured to be fitted to at least one of a shoulder region and a breast region of a user; a second harness configured to be fitted to a leg region or a waist region of the user; a belt body provided so as to extend to the first harness and to the second harness along the back side of the user; an actuator provided in the first harness and configured to wind and unwind a part of the belt body; a cover configured to cover the actuator; and a belt cover connected to the cover and configured to cover at least a part of the belt body.

With the assist device according to the second aspect of the present disclosure, the belt body is provided so as to extend to the first harness and to the second harness along the back side of a user. By the actuator winding the belt body, tension acts on the belt body. The tension causes generation of an assist force for assisting the user's work, which reduces a burden on the body of the user.

For example, when a user (caregiver) changes his/her posture from a forward-tilted posture to an upright posture while holding a load (care-receiver) with his/her hands, tension acts on the belt body by the actuator winding the belt body. The tension makes it easy for the user to change his/her posture from a forward-tilted posture to an upright posture and thus reduces a burden on the body of the user. In addition, the belt body is light in weight and can conform to the body of the user even if the user changes his/her posture, and thus follows movement of the user. Therefore, an assist device that provides good wear comfort can be provided.

Also, with the assist device having the above configuration, the cover and the belt cover can prevent contact between the actuator and a part of the belt body and the care-receiver when the assist device and the care-receiver come into contact with each other. Consequently, even if the assist device and the care-receiver come into contact with each other, operation of the assist device can be prevented from being hindered, and thus, the assist device can operate smoothly.

In the assist device according to the second aspect of the present disclosure, the belt cover may have a tubular shape including an opening at each of one end and another end, the one end may connect with the cover, and the opening at the other end may be provided below a position corresponding to the breast region on the back side of the user. The belt body may be connected from the first harness to the second harness via the opening at the other end.

For example, when a user (caregiver) raises a care-receiver in his/her arms, the care-receiver may cling to the user with his/her hands put around the back side of the user from the neck or underarms of the user. In the case of the assist device according to the second aspect of the present disclosure, the belt body is connected to the second harness via the opening at the other end, the opening being provided below the position corresponding to the breast region of the user on the back side of the user, and thus, the belt body is covered by the belt cover on the upper side relative to the opening. Therefore, even if the care-receiver puts his/her hands around the back side of the user, the care-receiver comes into contact with the cover or the belt cover of the assist device. Accordingly, operation of the assist device can be prevented from being hindered, and thus, the assist device can operate smoothly.

In the assist device according to the second aspect of the present disclosure, the belt cover may include a contractible portion configured to contract along with winding of the belt body. With the assist device according to the second aspect of the present disclosure, even if a part of the belt body is caught on the belt cover, winding of the belt body is not hindered because the belt cover contracts along with the winding of the belt body. Consequently, the assist device can operate more smoothly.

In the assist device according to the second aspect of the present disclosure, the belt body may include a first belt configured to be wound and unwound by the actuator, a second belt attached to the second harness and a joining member that joins the first belt and the second belt, and the belt cover may be configured to cover an entirety of a part of the first belt, the part being exposed from the cover.

With the assist device according to the second aspect of the present disclosure, by the actuator winding the first belt, tension acts on the first belt and the second belt. The tension causes generation of an assist force for assisting the user's work, which reduces a burden on the body of the user. Also, the belt cover covers the entirety of the part of the first belt, the part being exposed from the cover, and thus, contact between the belt body, which operates by being wound by the actuator, and the care-receiver can more reliably be prevented by the cover and the belt cover. Therefore, the assist device can operate smoothly.

The assist device according to the second aspect of the present disclosure provides good wear comfort and can operate smoothly when a user gives assistance.

### BRIEF DESCRIPTION OF THE DRAWINGS

Features, advantages, and technical and industrial significance of exemplary embodiments of the present disclosure will be described below with reference to the accompanying drawings, in which like numerals denote like elements, and wherein:

FIG. 1 is a back view illustrating an example of an assist device;

FIG. 2 is a back view of the assist device attached to the body of a user;

FIG. 3 is a side view of the assist device attached to the body of the user;

FIG. 4 is a diagram illustrating the user wearing the assist device in a forward-tilted posture;

FIG. 5 is a diagram of a control box and a belt body;

FIG. 6 is a diagram of a case where a user wearing the assist device changes his/her posture;

FIG. 7 is a back view of a first harness;

FIG. 8 is a side view illustrating another form of assist device;

FIG. 9 is a back view illustrating an assist device according to Embodiment 2;

FIG. 10 is a back view of the assist device according to Embodiment 2 attached to the body of a user;

FIG. 11 is a side view of the assist device according to Embodiment 2 attached to the body of the user;

FIG. 12 is a diagram illustrating the user wearing the assist device according to Embodiment 2 in a forward-tilted posture;

FIG. 13 is a diagram of a control box and a belt body in the assist device according to Embodiment 2;

FIG. 14 is a diagram of the control box and a belt cover in the assist device according to Embodiment 2;

FIG. 15A is a sectional view along a cutting-plate line indicated by arrow XV in FIG. 14;

FIG. 15B is a sectional view along the cutting-plate line indicated by arrow XV in FIG. 14;

FIG. 15C is a sectional view along the cutting-plate line indicated by arrow XV in FIG. 14;

FIG. 16 is a diagram of the control box and the belt cover in the assist device according to Embodiment 2;

FIG. 17 is a sectional view along a cutting-plate line indicated by arrow XVII in FIG. 16;

FIG. 18 is a diagram of a case where a user wearing the assist device according to Embodiment 2 changes his/her posture;

FIG. 19 is a diagram of a control box and a belt cover in an assist device according to an alteration;

FIG. 20 is a diagram of the control box and the belt cover in the assist device according to the alteration;

FIG. 21 is a diagram of a control box and a belt cover in an assist device according to an alteration; and

FIG. 22 is a diagram of the control box and the belt cover in the assist device according to the alteration.

### DETAILED DESCRIPTION OF EMBODIMENTS

### Embodiment 1

### Overall Configuration of Assist Device 10

FIG. 1 is a back view illustrating an example of an assist device. FIG. 2 is a back view of the assist device attached to the body of a user. FIG. 3 is a side view of the assist device attached to the body of the user. FIG. 4 is a diagram illustrating the user wearing the assist device having a forward-tilted posture (stooped posture). The assist device 10 illustrated in FIG. 1 includes one first harness 11 to be fitted to left and right shoulder regions BS, which are parts of the body of a user (human being), and two second harnesses 12 to be fitted to left and right leg regions BL, which are other parts of the body of the user.

The first harness 11 only needs to be fitted to at least either the shoulder regions BS or a breast region BC of the user, and also, may have a form other than the illustrated form. In Embodiment 1, the second harnesses 12 are fitted to respective knee regions BN in the leg regions BL. The second harnesses 12 also may have a form other than the illustrated form.

In the assist device 10 according to Embodiment 1, “left” and “right” are the user's own left and right when the user has an upright posture wearing the assist device 10 and “front” and “rear” are the user's own front and rear, “upper (up)” and “lower (down)” are the user's own upper and lower sides. “Upper (up)” are the head side of the user and “lower (down)” are the foot side of the user.

The assist device 10 includes a belt body 13, an actuator 14, a controller 15, a battery 37 and a sensor 38 in addition to the first harness 11 and the left and right second harnesses 12.

The first harness 11 is fitted to the shoulder region BS of the user. One second harness 12 is fitted to the left knee region BN of the user. The other second harness 12 is fitted to the right knee region BN of the user. The left second harness 12 and the right second harness 12 are bilaterally symmetrical and have a same configuration. The first harness 11 and the two second harnesses 12 are fitted to respective regions away from each other across the lumbar spine (waist region BW), which is a joint of the user, that is, the shoulder regions BS and the leg regions BL.

The first harness 11 includes a back body portion 21 to be fitted to the back of the user, and shoulder belts 22 and underarm belts 23 connecting with the back body portion 21. Each of the shoulder belts 22 and the underarm belts 23 is formed of, e.g., flexible fabric. The shoulder belts 22 and the underarm belts 23 allow the back body portion 21 to be held on the back of the user. The underarm belts 23 connect the back body portion 21 and the respective shoulder belts 22 and are adjustable in length. By adjustment of the length of each underarm belt 23, the back body portion 21 is brought into close contact with the user. The first harness 11 is fitted to the shoulder regions BS so as to be incapable of moving forward and backward, leftward and rightward, and upward and downward. The first harness 11 may include a stiff member, for example, as a part to be hung on the shoulder regions BS.

The second harnesses 12 are each formed of, e.g., flexible fabric. Each second harness 12 includes a knee body portion 24 to be fitted to the rear side of the relevant knee region BN of the user, and knee belts 25 provided so as to extend from the knee body portion 24. The knee belts 25 extend around the knee region BN at respective positions above and below the knee region BN, and the distal end side of each knee belt 25 is fixed to the knee body portion 24. The knee belt 25 can be adjusted in length of wrapping of the knee region BN, by a locking member such as a belt and a buckle or a hook-and-loop fastener. By the adjustment, the knee body portion 24 is brought into a close contact with the rear side of the knee region BN. The second harnesses 12 are fitted to the knee regions BN so as to be incapable of moving forward and backward, leftward and rightward, and upward and downward.

The belt body 13 is provided along the back side of the user so as to connect the first harness 11 and the second harnesses 12. The belt body 13 includes a first belt 16 provided on the upper body side, a second belt 17 provided on the lower body side, and a joining member 18 joining the first belt 16 and the second belt 17. Each of the first belt 16 and the second belt 17 is long and flexible. The joining member 18 is made of a metal and is formed by a rectangular ring-like body called “rectangular ring”.

Each of the first belt 16 and the second belt 17 is a band-like member made of fabric or leather (for example, a member formed of fabric obtained by flat knitting of polyester fibers) and is bendable along a shape of the body. Note that each of the first belt 16 and the second belt 17 may be a string-like belt (wire-like member). Each of the first belt 16 and the second belt 17 according to Embodiment 1 is a non-stretchable member, that is, has a characteristic of being hard to stretch in a longitudinal direction of the first belt 16 or the second belt 17 or a characteristic of not stretching in the longitudinal direction.

The assist device 10 according to Embodiment 1 includes a control box 30. The control box 30 is provided in the back body portion 21 of the first harness 11. FIG. 5 is a diagram of the control box 30 and the belt body 13. The control box 30 includes a plate-like base 31 and a cover 32 that covers the base 31. In order to describe an inner structure of the control box 30, in FIG. 5, the cover 32 is indicated by an imaginary line (alternate long and two short dashes line). In the assist device 10 according to Embodiment 1, the base 31 is attached to the back body portion 21 of the first harness 11.

The actuator 14, the controller 15, the battery 37, the sensor 38, etc., are provided in space between the base 31 and the cover 32. In the cover 32, an opening (cutout) 32a is formed and the first belt 16 extends through the opening 32a.

The actuator 14 is provided inside the control box 30. In other words, the actuator 14 is mounted on the base 31 and covered by the cover 32. The actuator 14 is provided on the first harness 11 via the base 31. In order to mount the actuator 14, the base 31 is formed of a stiff member. Note that where the back body portion 21 of the first harness 11 is formed of a particularly highly rigid member, the base 31 may be omitted and the actuator 14 may be mounted on the back body portion 21.

The actuator 14 enables winding and unwinding of a part of the belt body 13. For that purpose, the actuator 14 includes a motor 33, a reducer section 34 and a drive pulley 35. The motor 33 is a brushless DC motor. The motor 33 can rotate with a predetermined torque at a predetermined rotation frequency based on a drive signal output from the controller 15.

A parameter relating to rotation, such as a rotational angle, a rotation speed or a rotation frequency, of the motor 33 is detected by a rotation detector 36 attached to the motor 33. The rotation detector 36 according to Embodiment 1 is a rotary encoder but may be a Hall sensor or a resolver. A result of detection by the rotation detector 36 is input to the controller 15. By the controller 15 controlling operation of the motor 33 based on the result of detection, the assist device 10 can generate a proper assist force.

The reducer section 34 is formed by a plurality of gears, reduces the rotation frequency of the motor 33 and rotates an output shaft 34a of the reducer section 34. The drive pulley 35 is joined to the output shaft 34a and thus rotates together with the output shaft 34a. One end portion 16a side of the first belt 16 is attached to the drive pulley 35. Upon the drive pulley 35 rotating in one direction by forward rotation of the motor 33, the first belt 16 is wound on the drive pulley 35. Upon the drive pulley 35 rotating in the other direction, the first belt 16 is unwound from the drive pulley 35.

As above, the actuator 14 includes the drive pulley 35 that can wind the belt body 13, and the motor 33 for making the drive pulley 35 perform an operation of winding the belt body 13.

The controller 15 is formed by a control unit including a microcomputer. The controller 15 controls operation of the actuator 14 (motor 33). As the sensor 38, an acceleration sensor is provided. A signal from the sensor 38 is input to the controller 15. The controller 15 can estimate a posture of the user based on the signal from the sensor 38. The battery 37 supplies electric power to the controller 15, the motor 33, the rotation detector 36 and the sensor 38. The sensor 38 may be provided outside the control box 30.

### Belt Body 13

As described above, the belt body 13 includes a first belt 16, a second belt 17 and a joining member 18. One end portion 16a side of the first belt 16 is wound on the drive pulley 35 and is fixed. The other end portion 16b side of the first belt 16 is fixed to the joining member 18. Upon the first belt 16 being wound on the drive pulley 35, the joining member 18 is pulled up. Upon the joining member 18 being forcibly pulled down by a motion of the user (for example, a motion of taking a forward-tilted posture), the first belt 16 is unwound (pulled out) from the drive pulley 35. An amount of winding or unwinding (pullout) of the first belt 16 in the drive pulley 35 and an amount of rotation of an output shaft of the motor 33 are correlated with each other. A parameter relating to rotation of the motor 33 accompanying winding or unwinding of the belt body 13 is detected by the rotation detector 36.

As described above, the joining member 18 is formed by a rectangular ring-like body. A shaft portion 27a on one side (upper side) of the ring-like body is a first attachment portion 27, and the other end portion 16b of the first belt 16 is attached to the first attachment portion 27. In Embodiment 1, the first belt 16 is undetachable from the first attachment portion 27, but may be detachable by use of, e.g., a buckle.

The other side (lower side) of the rectangular ring-like body forming the joining member 18 is a second attachment portion 28 for attaching the second belt 17. As described above, the joining member 18 includes the first attachment portion 27 for attaching the first belt 16 and the second attachment portion 28 for attaching the second belt 17.

The second attachment portion 28 supports the second belt 17 in such a manner that the second belt 17 is folded at an intermediate point (intermediate portion 17c) of the second belt 17. In the assist device 10 according to Embodiment 1, the second attachment portion 28 includes a shaft portion 28a integrated with the first attachment portion 27, and a rotary pulley 29 rotatably supported on the shaft portion 28a. The second belt 17 is hung on the rotary pulley 29 so as to be folded at an intermediate point of the second belt 17. This configuration provides a configuration in which the second belt 17 is not fixed to the second attachment portion 28 but the second belt 17 is supported so as to be movable in both directions in the longitudinal direction (arrow X directions in FIG. 5) in a state in which the second belt 17 is folded.

In FIG. 2, the second belt 17 is attached to the second harnesses 12. More specifically, the second belt 17 is formed of a single band-like member. One end portion 17a side of the second belt 17 is attached to the left second harness 12. The other end portion 17d side of the second belt 17 is attached to the right second harness 12. As above, the intermediate portion 17c of the second belt 17 is put on the joining member 18.

According to the above configuration of the second belt 17, the second belt 17 includes a left second belt portion 19 extending from the joining member 18 to the left second harness 12 and a right second belt portion 20 extending from the joining member 18 to the right second harness 12. As above (see FIG. 5), the second belt 17 is hung on the second attachment portion 28 (rotary pulley 29) and is not fixed, and thus, a length of the left second belt portion 19 and a length of the right second belt portion 20 can freely be changed. However, a total of the length of the left second belt portion 19 and the length of the right second belt portion 20 is fixed. This configuration prevents, for example, walking of the user from being restricted by the second belt 17, and thus, enables the user to easily walk.

The second belt 17 further includes a connecting member 39 connecting the left second belt portion 19 and the right second belt portion 20. For example, where the user changes his/her posture from an upright posture to a stooped posture as illustrated in FIG. 4, the connecting member 39 enables preventing an increase in lateral distance between the left second belt portion 19 and the right second belt portion 20. In other words, the connecting member 39 enables preventing a failure of the left second belt portion 19 and the right second belt portion 20 to extend along the respective back sides of the leg regions BL of the user. The connecting member 39 is a band-like member.

Although not illustrated, the connecting member 39 and the left second belt portion 19 are joined via, e.g., a pin so as to be rotatable relative to each other, and although not illustrated, the connecting member 39 and the right second belt portion 20 are joined via, e.g., a pin so as to be rotatable relative to each other. Consequently, each of the left second belt portion 19 and the right second belt portion 20 is not prevented from moving in both longitudinal directions (arrow X directions in FIG. 5).

As illustrated in FIG. 2, the one end portion 17a side of the second belt 17 may be attached to the left second harness 12 via a fastener 40 such as a buckle. Also, the other end portion 17d side of the second belt 17 may be attached to the right second harness 12 via a fastener 40 such as a buckle. Consequently, the second belt 17 is detachable from the second harnesses 12.

### Sensor 38 and Controller 15

In FIG. 5, as described above, the sensor 38 is formed by an acceleration sensor. The controller 15 can perform various types of arithmetic processing. By the controller 15 performing arithmetic processing of a signal from the sensor 38, a motion and a posture of the user can be detected. The sensor 38 has a configuration that outputs a signal according to a posture of the user, and thus functions as a posture detector for detecting a posture of the user. For example, it is possible to detect whether a posture of the upper body of the user is a forward-tilted posture or an upright posture or detect that the user has taken a crouching posture.

Also, an amount of winding and unwinding of the belt body 13 in the drive pulley 35 by the motor 33 and a posture of the user are correlated with each other. Therefore, the controller 15 can estimate a posture of the user based on a rotational angle of the motor 33 detected by the rotation detector 36. The rotation detector 36 functions as a posture detector for detecting a posture of the user.

The controller 15 processes a signal from one or each of both of the sensor 38 and the rotation detector 36, and outputs a drive signal to the actuator 14 (motor 33) based on a result of the processing, that is, the posture of the user. Based on the drive signal, the actuator 14 (motor 33) operates to, e.g., wind and unwind the belt body 13 and temporarily stop the winding and unwinding.

In a state in which the assist device 10 is worn by the user, the motor 33 constantly operates (generates torque) in a direction in which the belt body 13 is wound, with a small force that is smaller than that of a case where the motor 33 generates an assist force, according to the control by the controller 15, to cause generation of small tension in the belt body 13. Consequently, the belt body 13 is not loosened.

Upon the user changing his/her posture, for example, from an upright posture to a forward-tilted posture, tension is generated in the belt body 13 because of the posture change. Therefore, in this case, upon a start of the posture change to a forward-tilted posture, the motor 33 is forcibly rotated (the motor 33 idles) by the tension of the belt body 13 irrespective of power of the actuator 14, and the belt body 13 is thereby unwound. Alternatively, upon a start of the posture change to a forward-tilted posture, the actuator 14 operates, that is, the motor 33 is driven to rotate, to unwind the belt body 13.

On the other hand, upon the user changing his/her posture from a forward-tilted posture to an upright posture, the belt body 13 is about to be loosened because of the posture change. Therefore, in this case, upon a start of the posture change to an upright posture, in order to maintain tension acting on the belt body 13, the actuator 14 operates, that is, the motor 33 is driven to rotate, to wind the belt body 13.

In this way, by the user's posture change, the belt body 13 is wound or unwound. In the winding or unwinding, the motor 33 actively or passively rotates by a predetermined rotational angle. The rotational angle at this time is detected by the rotation detector 36. In this way, an amount of operation of the actuator 14 (motor 33) in winding or unwinding of the belt body 13 due to the user's posture change is detected by the rotation detector 36. Then, the controller 15 can acquire the amount of operation of the actuator 14 (rotational angle of the motor 33) in winding or unwinding of the belt body 13 due to the user's posture change, and based on the amount of operation, perform control of operation of the actuator 14 in order to provide an assist force to the user.

### Assist Force of Assist Device 10

FIG. 6 is a diagram of a case where the user wearing the assist device 10 changes his/her posture. The assist device 10 can provide an assist force to the user for the posture change. The below description relating to the assist force will be provided in terms of a case where a caregiver who is a user of the assist device 10 assists a change in posture of a care-receiver who is a person other than the user by, e.g., raising the care-receiver in his/her arms.

Upon the first belt 16 being wound on the drive pulley 35 by the motor 33 of the actuator 14, the joining member 18 pulls the second belt 17 up toward the actuator 14 side, that is, the upper side. The opposite end portions 17a, 17d of the second belt 17 are attached to the left and right second harnesses 12, respectively. The second harnesses 12 are fixed to the respective knee regions BN. Therefore, upon the first belt 16 being wound on the drive pulley 35, tension acts on the first belt 16 and the second belt 17. The tension acts as an assist force for the caregiver.

A case where the caregiver changes his/her posture from an upright posture to a forward-tilted posture will be described. Upon a start of the posture change to a forward-tilted posture, the belt body 13 is unwound against the small force. Consequently, the caregiver can effortlessly take a forward-tilted posture. Upon a forward-tilting angle of the upper body of the caregiver relative to a vertical line reaching θ and the caregiver stopping at the forward-tilting angle of θ, the unwinding of the belt body 13 is stopped. Note that a start and an end of a posture change can be detected by the rotation detector 36 or the sensor 38.

Upon the caregiver starting to change his/her posture in a direction from a forward-tilted posture to an upright posture to raise a care-receiver in his/her arms, the actuator 14 winds the belt body 13 with a force that is larger than the small force. Consequently, great tension is generated in the belt body 13. The tension causes generation of a rearward acting force F1 in the first harness 11. In other words, an acting force F1 is generated in a direction in which the upper body of the caregiver having a forward-tilted posture is raised. Also, simultaneously, in the second belt 17, the tension causes generation of an acting force F2 pushing a left hip region and a right hip region of the caregiver forward. Consequently, the caregiver can easily return from the forward-tilted posture to an upright posture.

Also, where the caregiver takes a bent posture (crouching posture) in which the upper body is tilted forward and knee regions are bent as illustrated in FIG. 4, the assist device 10 can provide an assist force to the caregiver. Where the caregiver changes his/her posture from a bent posture to an upright posture, for example, where the caregiver wearing the assist device 10 lifts up a part of the body of the care-receiver, the actuator 14 winds the belt body 13. Consequently, tension is generated in the belt body 13.

The tension causes generation of a rearward acting force F1 in the first harness 11. In other words, an acting force F1 in a direction in which the upper body of the caregiver having a forward-tilted posture is raised is generated. Also, simultaneously, the tension causes generation of an acting force F2 pushing the left and right hip regions of the caregiver forward in the second belt 17. Furthermore, a rearward acting force F3 is generated in the second harness 12. The above acting forces F1, F2, F3 reduce a load on muscles such as the back muscle, the quadriceps, etc., of the caregiver having a stooped posture and thus enables assisting a motion of lifting up a load.

The assist device 10 also functions in a case where the caregiver changes his/her posture from an upright posture to a bent posture. Examples of the case include a case where the caregiver lifts down a part of the body of the care-receiver. In this case, the actuator 14 unwinds the belt body 13 while exerting a braking force for the unwinding of the belt body 13. In other words, the motor 33 rotates in a direction in which the belt body 13 is unwound, but torque in the winding direction is generated in the motor 33. Consequently, tension is generated in the belt body 13. In this case, also, the assist device 10 enables reducing a load on muscles such the back muscle, the quadriceps, etc., of the caregiver in a stooped posture and thus enabling assisting a lift-down motion by means of the above acting forces F1, F2, F3. As above, the assist device 10 according to Embodiment 1 enables reducing a load on the muscles of the waist region in a stooped posture and preventing lower back pain.

Where the caregiver maintains a forward-tilted posture, also, the assist device 10 according to Embodiment 1 enables easily maintaining the posture. In other words, as illustrated in the figure on the right side of FIG. 6, in a state in which the caregiver has taken a first forward-tilted posture, the operation of the actuator 14 stops to prevent unwinding of the belt body 13. Even if the caregiver attempts to take a further forward-tilted posture (second forward-tilted posture), tension of the belt body 13 connecting the first harness 11 and the second harnesses 12 prevents taking the second forward-tilted posture. In other words, the assist device 10 attempts to maintain the first forward-tilted posture of the caregiver. For the caregiver, it is easy to maintain the first forward-tilted posture. As a result, for example, where the caregiver continues having the first forward-tilted posture for a long time for work, a load on the body is reduced.

### Grip Portion 60 Included in First Harness 11

FIG. 7 is a back view of the first harness 11. FIG. 7 illustrates a state in which the control box 30 has been detached from the first harness 11. Grip portions 60 to be held by a care-receiver (person other than a user) are attached to the first harness 11. In the assist device 10 according to Embodiment 1, as described above, the first harness 11 includes the back body portion 21 that faces the back of the user, and the shoulder belts 22 for fixing the first harness 11 to the upper body of the user, the shoulder belts 22 connecting with the back body portion 21. The first harness 11 further includes the underarm belts 23 and the fixing of the first harness 11 to the user is assisted by the underarm belts 23.

As illustrated in FIG. 7, the grip portions 60 are attached to the back body portion 21. Like the shoulder belts 22, the back body portion 21 may be formed of, e.g., flexible fabric but is preferably formed of a stiff member having a rigidity that is higher than that of the shoulder belts 22. For example, the back body portion 21 is made of a resin (bubble polyethylene). The grip portions 60 to be held by a care-receiver are attached to the back body portion 21 and a load acts on the back body portion 21 through the grip portions 60. Therefore, the back body portion 21 is preferably a stiff member having a high rigidity (strength).

The grip portions 60 are provided on the opposite, left and right, sides of the first harness 11. Each grip portion 60 is a belt-like member and opposite ends of the grip portion 60 is attached to the back body portion 21. More specifically, one end 60a side of the left grip portion 60 is connected to an upper portion 51L on the left side of the back body portion 21. The other end 60b side of the left grip portion 60 is connected to a lower portion 52L on the left side of the back body portion 21. Consequently, the left grip portion 60 forms a loop shape jointly with the back body portion 21. One end 60c side of the right grip portion 60 is connected to an upper portion 51R on the right side of the back body portion 21. The other end 60d side of the right grip portion 60 is connected to a lower portion 52R on the right side of the back body portion 21. Consequently, the right grip portion 60 forms a loop shape jointly with the back body portion 21.

In the assist device 10 according to Embodiment 1, as described above, the actuator 14 is provided inside the control box 30. In other words, the actuator 14 is mounted on the base 31 (see FIG. 5) and covered by the cover 32. The cover 32 is attached to the base 31. At the time of maintenance of the actuator 14, the cover 32 is detached from the base 31.

The actuator 14 includes the motor 33, the reducer section 34 and the drive pulley 35. The motor 33, the reducer section 34 and the drive pulley 35 are attached to the base 31. The battery 37, the sensor 38 and the controller 15 are also attached to the base 31. The base 31 is attached to the back body portion 21 of the first harness 11.

The base 31 is preferably detachably attached to the back body portion 21. For example, a projection 46 (see FIG. 7) is provided at one of the base 31 and the back body portion 21 and a recess portion 47 that is engageable with the projection 46 is provided in the other of the base 31 and the back body portion 21. The control box 30 is attached to the back body portion 21 by engaging the projection 46 with the recess portion 47. The control box 30 is detached from the back body portion 21 by disengaging the projection 46 and the recess portion 47 from each other.

As described above, the actuator 14 is mounted on the base 31 that is separate from the back body portion 21. The base 31 is detachably attached to the back body portion 21. Then, the grip portions 60 are attached not to the base 31 but to the back body portion 21. Note that a configuration for making the control box 30 attachable/detachable to/from the back body portion 21 may be a configuration other than the above configuration using the projection 46 and the recess portion 47.

As illustrated in FIG. 7, a lug portion 55 that is held by a person (the caregiver or a third person) who performs work for the detachment is provided in the first harness 11 when the first harness 11 is detached from a caregiver who is a user of the assist device 10. At the time of handling the first harness 11 during and after detachment of the first harness 11 from the caregiver, the lug portion 55 is held. The lug portion 55 is attached to an upper portion of the back body portion 21. The lug portion 55 is also a belt-like member like the grip portions 60.

### Assist Device 10 According to Embodiment 1

As above, the assist device 10 according to Embodiment 1 (see FIG. 2) includes the first harness 11 to be fitted to shoulder regions BS of a caregiver, the second harnesses 12 to be fitted to left and right leg regions BL of the caregiver, respectively, the belt body 13, and the actuator 14. The belt body 13 is provided so as to extend to the first harness 11 and to the second harnesses 12 along the back side of the caregiver. The actuator 14 is provided in the first harness 11 and is configured to be capable of winding and unwinding a part of the belt body 13.

With this assist device 10, the belt body 13 is provided so as to extend to the first harness 11 and to the second harnesses 12 along the back side of a caregiver. By the actuator 14 winding the belt body 13 (first belt 16), tension acts on the first belt 16 and the second belt 17. The tension causes generation of an assist force for assisting the caregiver's work, which reduces a burden on the body of the caregiver.

For example, when a caregiver changes his/her posture from a forward-tilted posture to an upright posture (see FIG. 6) while holding a care-receiver in his/her arms, tension acts on the belt body 13 by the actuator 14 winding the belt body 13. The tension makes it easy for the caregiver to change his/her posture from a forward-tilted posture to an upright posture and thus reduces a burden on the body of the caregiver. In other words, tension acting on the belt body 13 is generated by the actuator 14 as an assist force. Also, the belt body 13 according to Embodiment 1 is non-stretchable over an entire length thereof. Consequently, a relationship between an output of the actuator 14 and tension acting on the belt body 13 becomes clear, which makes it easy to control a necessary assist force.

Then, as illustrated in FIG. 7, the assist device 10 according to Embodiment 1 includes the grip portions 60 to be held by a care-receiver, the grip portions 60 being attached to the first harness 11. For example, when a caregiver wearing the assist device 10 changes his/her posture from a forward-tilted posture to an upright posture while holding a care-receiver in his/her arms in order to raise the care-receiver, the care-receiver tries to seize the caregiver. In this case, letting the care-receiver hold the grip portions 60 enables preventing the belt body 13 from being held and operation of the belt body 13 is thus not hindered. Therefore, the assist device 10 properly operates.

In the assist device 10 according to Embodiment 1, the grip portions 60 are attached to the back body portion 21 included in the first harness 11. Therefore, the grip portions 60 are on the rear side of the first harness 11, that is, on the back side of the caregiver. Therefore, where the care-receiver flings his/her arms around the upper body of the caregiver, the care-receiver can easily hold the grip portions 60.

Also, the opposite end portions of the grip portions 60 are connected to the respective upper portions (51L, 51R) and the respective lower portions (52L, 52R) on the left and right sides of the back body portion 21 and the grip portions 60 each form a loop shape jointly with the back body portion 21. This configuration allows the care-receiver to easily hold the grip portions 60. Alternatively, the care-receiver can push his/her body against the caregiver by putting his/her arms between the grip portions 60 and the back body portion 21 forming the loop shapes. Also, forces acting on the left and right grip portions 60 as a result of the care-receiver holding the left and right grip portions 60 are transmitted to the back body portion 21 in such a manner that the forces are dispersed upward and downward. Therefore, even if the care-receiver holds the grip portions 60, the first harness 11 is stably fitted to the caregiver.

The actuator 14 is mounted on the base 31 and the base 31 is a member that is separate from the back body portion 21. The base 31 is attached to the back body portion 21 and the grip portions 60 are attached to the back body portion 21. With this configuration, forces acting on the grip portions 60 as a result of the care-receiver holding the grip portions 60 are transmitted to the first harness 11 through the back body portion 21. On the other hand, the forces acting on the grip portions 60 are less likely to act on the actuator 14. Therefore, operation such as winding of the belt body 13 by the actuator 14 is not hindered by the forces acting on the grip portions 60.

### Other Assist Devices 10

In the assist device 10 according to Embodiment 1 above, the second harnesses 12 are fitted to the leg regions BL of the user (caregiver). As illustrated in FIG. 8, a second harness 12 may be fitted to a waist region BW of a user. In this case, the second harness 12 may have the shape of a waist belt or the shape of pants. Where a second harness 12 is fitted to a waist region BW, an actuator 14 may be attached to a first harness 11 or may be attached to the second harness 12. In FIG. 8 an actuator 14 is attached to a first harness 11.

In the case of the assist device 10 illustrated in FIG. 8, also, a belt body 13 is provided so as to extend to the first harness 11 and to the second harness 12 along the back side of the user (caregiver). By the actuator 14 winding the belt body 13, tension acts on the belt body 13. The tension causes generation of an assist force for assisting the user's work, which reduces a burden on the body of the user. Then, the grip portions 60 to be held by a care-receiver are provided in the first harness 11. Although not illustrated, grip portions 60 may also be provided in the second harnesses 12. The grip portions 60 may be attached to at least either the first harness 11 or the second harnesses 12 according to the forms and fitted positions of the first harness 11 and the second harnesses 12.

For example, where the user (caregiver) raises a care-receiver while holding the care-receiver in his/her arms, the care-receiver holds not the control box 30 (actuator 14) but the grip portions 60 attached to the first harness 11. For that purpose, the grip portions 60 are provided at respective positions that allow the care-receiver to easily hold the grip portions 60. In other words, when the care-receiver puts his/her hands around the back side of the user, the hands of the care-receiver are often located on or close to the user's sides. The grip portions 60 attached to the first harnesses 11 are located at the respective positions on the left and right sides relative to the control box 30 and close to the user's sides. Therefore, the care-receiver can hold the grip portions 60 without extending his/her hands to the center of the back of the user.

### Embodiment 2

### Overall Configuration of Assist Device 10

An assist device according to Embodiment 2 will be described appropriately with reference to FIGS. 9 to 12. FIG. 9 is a back view illustrating an assist device according to Embodiment 2. FIG. 10 is a back view of the assist device according to Embodiment 2 attached to the body of a user. FIG. 11 is a side view of the assist device according to Embodiment 2 attached to the body of the user. FIG. 12 is a diagram illustrating the user wearing the assist device according to Embodiment 2 having a forward-tilted posture (stooped posture). Note that components that are the same as the components in FIGS. 1 to 4, which illustrate Embodiment 1, are provided with reference numerals that are the same as those in FIGS. 1 to 4 and description of such components may be omitted.

The assist device 10 according to Embodiment 2 includes a control box 30 and a belt cover 50. The control box 30 is provided in a back body portion 21 of a first harness 11. The belt cover 50 is a cover that is connected to the control box 30 and covers a part of a belt body 13.

FIG. 13 is a diagram of the control box 30 and the belt body 13 in the assist device 10 according to Embodiment 2. Note that components that are the same as the components in FIG. 5, which illustrates Embodiment 1, are provided with reference numerals that are the same as those in FIG. 5 and description of such components may be omitted. Also, FIG. 14 is a diagram of the control box 30 and the belt cover 50 in the assist device 10 according to Embodiment 2. FIGS. 15A to 15C are sectional views along a cutting-plate line indicated by arrow XV in FIG. 14, FIG. 15A is a sectional view according to Embodiment 2, FIGS. 15B and 15C are sectional views each illustrating an alteration of Embodiment 2.

A description will be provided with reference to FIG. 13. The control box 30 includes a plate-like base 31 and a cover 32 that covers the base 31. In order to describe an inner structure of the control box 30, in FIG. 13, the cover 32 is indicated by an imaginary line (alternate long and two short dashes line). In Embodiment 2, the base 31 functions as a back body portion 21 of the first harness 11.

An actuator 14, a controller 15, a battery 37, a sensor 38, etc., are provided in space between the base 31 and the cover 32. In other words, the cover 32 covers the actuator 14, the controller 15, the battery 37 and the sensor 38 from behind a user. In the cover 32, an opening (cutout) 32a is formed and a first belt 16 extends through the opening 32a.

The actuator 14 is provided inside the control box 30. In other words, the actuator 14 is provided in the first harness 11. The actuator 14 enables winding and unwinding a part of the belt body 13. For that purpose, the actuator 14 includes a motor 33, a reducer section 34 and a drive pulley 35. The motor 33 is a brushless DC motor. The motor 33 can rotate with a predetermined torque at a predetermined rotation frequency based on a drive signal output from the controller 15. The motor 33 can rotate forward and reversely based on a drive signal output from the controller 15.

A parameter relating to rotation, such as a rotational angle, a rotation speed or a rotation frequency, of the motor 33 is detected by a rotation detector 36 attached to the motor 33. The rotation detector 36 according to Embodiment 2 is a rotary encoder but may be a Hall sensor or a resolver. A result of detection by the rotation detector 36 is input to the controller 15. By the controller 15 controlling operation of the motor 33 based on the result of detection, the assist device 10 can generate a proper assist force.

The reducer section 34 is formed by a plurality of gears, reduces the rotation frequency of the motor 33 and rotates an output shaft 34a of the reducer section 34. The drive pulley 35 is joined to the output shaft 34a and thus rotates together with the output shaft 34a. One end portion 16a side of the first belt 16 is attached to the drive pulley 35. Upon the drive pulley 35 rotating in one direction by forward rotation of the motor 33, the first belt 16 is wound on the drive pulley 35. Upon the drive pulley 35 rotating in the other direction by reverse rotation of the motor 33, the first belt 16 is unwound from the drive pulley 35.

As above, the actuator 14 includes the drive pulley 35 that can wind the belt body 13, and the motor 33 for making the drive pulley 35 perform an operation of winding the belt body 13. The first belt 16 is wound and unwound by the actuator 14.

The controller 15 is formed by a control unit including a microcomputer. The controller 15 controls operation of the actuator 14 (motor 33). The sensor 38 includes an acceleration sensor. A signal detected by the sensor 38 is input to the controller 15. The controller 15 includes an arithmetic section and performs an arithmetic operation based on the signal from the sensor 38 to estimate a posture of the user. The battery 37 supplies electric power to the controller 15, the motor 33, the rotation detector 36 and the sensor 38. Note that the sensor 38 may be provided outside the control box 30.

A description will be described with reference to FIG. 14. In order to describe respective outer structures of the control box 30 and the belt cover 50, in FIG. 14, the base 31 and a part of the belt body 13 extending through the inside of the belt cover 50 are indicated by imaginary lines (dashed lines). The belt cover 50 is formed by a molded product (product molded using injection molding) made of a synthetic resin (for example, 6, 6-nylon) and a surface of the belt cover 50 has a flat shape. The belt cover 50 has a tubular shape including an opening at each of one end and the other end.

The one end of the belt cover 50 is connected to the cover 32 and an upper opening 501 formed at the one end connects with the opening 32a of the cover 32. A lower opening 502 formed at the other end is provided below a position corresponding to a breast region BC on the back side of the user. In Embodiment 2, the lower opening 502 is located in a back BB below the position corresponding to the breast region BC on the back side of the user. Note that in carrying out the present disclosure, the location of the lower opening 502 is not limited to this example but the lower opening 502 may be located in a waist region BW on the back side of the user.

As illustrated in FIGS. 14 and 15A, a part of the belt body 13 is provided so as to extend through the inside of the tubular belt cover 50. FIGS. 14 and 15A illustrates a state in which a part of the belt body 13 is unwound by the actuator 14 and a joining member 18 comes out below the lower opening 502. A width of the inside of the belt cover 50 is at least larger than a width of the part of the belt body 13 and the belt body 13 can move upward and downward inside the belt cover 50. The belt body 13 is connected from the first harness 11 to second harnesses 12 via the lower opening 502.

Also, as illustrated in FIG. 15A, the belt cover 50 has a tubular shape and covers a part of the belt body 13 (first belt 16 in FIG. 15A) from behind and also from the back BB of the user. In other words, a part of the belt body 13 is received in the belt cover 50. Here, since the surface of belt cover 50 has a flat shape, the surface of belt cover 50 can come into smooth contact with the user's clothing in the back BB of the user. The belt cover 50 slides on the user's clothing along with movement of the user, enabling provision of an assist device 10 that provides good wear comfort to a user.

FIG. 16 is a diagram of the control box 30 and the belt cover 50 in the assist device 10 according to Embodiment 2. FIG. 17 is a sectional view along a cutting-plate line indicated by arrow XVII in FIG. 16. FIGS. 16 and 17 illustrate a state in which a part of the belt body 13 is wound by the actuator 14, the joining member 18 is located above the lower opening 502, the entire first belt 16 and the entire joining member 18 and a part of a second belt 17 are received in the belt cover 50.

In the case of the assist device 10 according to Embodiment 2, when a care-receiver seizes the back BB of the user, the care-receiver comes into contact with the cover 32 or the belt cover 50, enabling preventing contact between the care-receiver and the belt body 13. Since a part of the belt body 13 is wound and unwound by the actuator 14, preventing contact with the belt body 13 that operates in such a manner enables the assist device 10 to operate more smoothly.

In particular, when the user (caregiver) raises the care-receiver in his/her arms, the care-receiver may cling to the user with his/her hands put around the back side of the user from the neck or underarms of the user. In the case of the assist device 10 according to Embodiment 2, the belt body 13 is connected to the second harnesses 12 via the lower opening 502 provided below the position corresponding to the breast region BC on the back side of the user, and thus, even if the care-receiver puts his/her hands around the back side of the user, the care-receiver comes into contact with the cover 32 or the belt cover 50 located above the lower opening 502 in the assist device 10. Therefore, operation of the assist device 10 can be prevented from being hindered, and thus, the assist device 10 can operate smoothly.

Note that the belt cover 50 according to Embodiment 2 has a tubular shape, but in carrying out the present disclosure, the shape of the belt cover is not limited to this example and the belt cover may have another shape as long as such shape is a shape that covers a part of the belt body 13 from behind the user. For example, instead of the belt cover 50, a belt cover 51 or a belt cover 52 having a sectional shape illustrated in FIG. 15B or 15C may be provided.

FIG. 15B is a diagram illustrating a section of a belt cover 51 according to an alteration of Embodiment 2, the section being similar to that in FIG. 15A. As illustrated in FIG. 15B, the belt cover 51 has a U-shape in a section and covers a part of a belt body 13 from behind a user. Since a part of the belt body 13 is received in space formed by the belt cover 51 and a back BB of the user, the belt cover 51 can prevent contact between a care-receiver and the belt body 13. Therefore, an assist device 10 according to the present alteration can operate smoothly.

FIG. 15C is a diagram illustrating a section of a belt cover 52 according to an alteration of Embodiment 2, the section being similar to that in FIG. 15A. As illustrated in FIG. 15C, the belt cover 52 has a plate-like shape in a section and covers a part of a belt body 13 from behind a user. A width of the belt cover 52 is larger than that of the part of the belt body 13. The belt cover 52 connects with a cover 32 and is supported behind the user by the cover 32. Since the part of the belt body 13 is interposed between the belt cover 52 and the back BB of the user, the belt cover 52 can prevent contact between a care-receiver and the belt body 13. Therefore, an assist device 10 according to the present alteration can operate smoothly.

A description will be provided with reference to FIG. 9. The second belt 17 is attached to the second harnesses 12. More specifically, the second belt 17 is formed of a single band-like member. One end portion 17a side of the second belt 17 is attached to the left second harness 12. The other end portion 17d side of the second belt 17 is attached to the right second harness 12. In the below, a part, from the joining member 18 to the left second harness 12, of the second belt 17 is referred to as “left second belt portion 19” and a part, from the joining member 18 to the right second harness 12, of the second belt 17 is referred to as “right second belt portion 20”.

In Embodiment 2, the one end portion 17a side of the second belt 17 is attached to the left second harness 12 via a fastener 40 (for example, a buckle). Also, the other end portion 17d side of the second belt 17 is attached to the right second harness 12 via a fastener 40 (for example, a buckle). Consequently, the second belt 17 is detachable from the second harness 12.

The second belt 17 includes a connecting member 39 connecting the left second belt portion 19 and the right second belt portion 20. The connecting member 39 connects the left second belt portion 19 and the right second belt portion 20 at respective intermediate positions between a folded portion (intermediate portion 17c) of the second belt 17 and fixation parts (the one end portion 17a and the other end portion 17d) fixed to the two second harnesses 12. The folded portion is a part in which the second belt 17 is folded in the joining member 18. The fixation parts are parts in which the second belt 17 is fixed to the two second harnesses 12, respectively.

For example, where the user changes his/her posture from an upright posture to a stooped posture, as illustrated in FIG. 12, the connecting member 39 enables preventing an increase in lateral distance between the left second belt portion 19 and the right second belt portion 20. In other words, the connecting member 39 enables preventing a failure of the left second belt portion 19 and the right second belt portion 20 to extend along the respective back sides of leg regions BL of the user.

A description will be provided with reference to FIG. 13. The second belt 17 is hung on a second attachment portion 28 (rotary pulley 29) and is not fixed, and thus, a length of the left second belt portion 19 and a length of the right second belt portion 20 can freely be changed. However, a total of the length of the left second belt portion 19 and the length of the right second belt portion 20 is fixed. This configuration prevents, for example, walking of the user from being restricted by the second belt 17, and thus, enables the user to easily walk.

### Assist Force of Assist Device 10

FIG. 18 is a diagram of a case where a user wearing the assist device 10 changes his/her posture. The assist device 10 can provide an assist force to the user for the posture change.

Upon the first belt 16 being wound on the drive pulley 35 by the motor 33 of the actuator 14, the joining member 18 pulls the second belt 17 up toward the actuator 14 side, that is, the upper side. The opposite end portions 17a, 17d of the second belt 17 are attached to the left and right second harnesses 12, respectively. The second harnesses 12 are fixed to respective knee regions BN. Therefore, upon the first belt 16 being wound on the drive pulley 35, tension acts on the first belt 16 and the second belt 17. The tension acts as an assist force for the user.

A case where the user changes his/her posture from an upright posture to a forward-tilted posture will be described. Upon a start of the posture change to a forward-tilted posture, the actuator 14 unwinds the belt body 13. Alternatively, the belt body 13 is unwound irrespective of power of the actuator 14. Consequently, the user can effortlessly take a forward-tilted posture. Upon a forward-tilting angle of the upper body of the user relative to a vertical line reaching θ and the user stopping at the tilting angle of θ, the unwinding of the belt body 13 is stopped. Note that a start and an end of a posture change can be detected by the rotation detector 36 or the sensor 38.

Upon the user starting to change his/her posture in a direction from a forward-tilted posture to an upright posture, the actuator 14 winds the belt body 13. Consequently, tension is generated in the belt body 13. The tension causes generation of a rearward acting force F1 in the first harness 11. In other words, an acting force F1 is generated in a direction in which the upper body of the user having a forward-tilted posture is raised. Also, simultaneously, in the second belt 17, an acting force F2 pushing a left hip region and a right hip region of the user forward is generated by the tension. Consequently, the user can easily return from the forward-tilted posture to an upright posture.

Also, where the user takes a bent posture (crouching posture) in which the upper body is tilted forward and knee regions are bent as illustrated in FIG. 12, the assist device 10 can provide an assist force to the user. Where the user changes his/her posture from a bent posture to an upright posture, for example, where the user lifts up an object or a part of the body of a care-receiver, the actuator 14 winds the belt body 13. Consequently, tension is generated in the belt body 13.

The tension causes generation of a rearward acting force F1 in the first harness 11. In other words, an acting force F1 in a direction in which the upper body of the user having a forward-tilted posture is raised is generated. Also, simultaneously, the tension causes generation of an acting force F2 pushing the left and right hip regions of the user forward in the second belt 17. Furthermore, a rearward acting force F3 is generated in the second harnesses 12. The above acting forces F1, F2, F3 reduce a load on muscles such as the back muscle, the quadriceps, etc., of the user having a stooped posture and thus enables assisting a motion of lifting up a load.

The assist device 10 also functions where the user changes his/her posture from an upright posture into a bent posture, for example, where the user lifts down an object or a part of the body of a care-receiver. In this case, the actuator 14 unwinds the belt body 13 while exerting a braking force to the unwinding of the belt body 13. In other words, the motor 33 rotates in a direction in which the belt body 13 is unwound, but torque in the winding direction is generated in the motor 33. Consequently, tension is generated in the belt body 13. In this case, also, the assist device 10 enables reducing a load on muscles such the back muscle, the quadriceps, etc., of the user in a stooped posture and thus enabling assisting a lift-down motion by means of the above acting forces F1, F2, F3. As above, the assist device 10 according to Embodiment 2 enables reducing a load on the muscles of the waist region in a stooped posture and preventing lower back pain.

Also, according to the assist device 10 according to Embodiment 2, even if the user has a bent posture with one of the left and right leg regions BL on the front side and the other on the rear side (in a laterally asymmetrical state), one (on the side on which the leg region is put forward) of the left second belt portion 19 and the right second belt portion 20 of the second belt 17 can automatically become longer than the other. Upon the first belt 16 being wound by the actuator 14 in this state, tension also acts on the second belt 17, and the tension acts on both of the left second belt portion 19 and the right second belt portion 20 and thus the tension is not relieved. Therefore, as described above, even if the user has a laterally asymmetrical posture, the assist device 10 according to Embodiment 2 enables a proper assist force to act on the user.

Where the user maintains a forward-tilted posture, also, the assist device 10 according to Embodiment 2 enables easily maintaining the posture. In other words, as illustrated in the figure on the right side of FIG. 18, in a state in which the user has taken a first forward-tilted posture, the operation of the actuator 14 stops to prevent unwinding of the belt body 13. Even if the user attempts to take a further forward-tilted posture (second forward-tilted posture), tension of the belt body 13 connecting the first harness 11 and the second harnesses 12 prevents taking the second forward-tilted posture. In other words, the assist device 10 attempts to maintain the first forward-tilted posture of the user. For the user, it is easy to maintain the first forward-tilted posture. As a result, for example, where the user continues having the first forward-tilted posture for a long time for work, a load on the body can be reduced.

As above, the assist device 10 according to Embodiment 2 (see FIG. 10) includes the first harness 11 to be fitted to shoulder regions BS of a user, the second harnesses 12 to be fitted to left and right leg regions BL of the user, respectively, the belt body 13, and the actuator 14. The belt body 13 is provided so as to extend to the first harness 11 and to the second harnesses 12 along the back side of the user. The actuator 14 is provided in the first harness 11 and is configured to be capable of winding and unwinding a part of the belt body 13.

The belt body 13 includes the first belt 16 to be wound and unwound by the actuator 14, the second belt 17 attached to the second harnesses 12, and the joining member 18 joining the first belt 16 and the second belt 17.

According to this assist device 10, the belt body 13 is provided so as to extend to the first harness 11 and to the second harnesses 12 along the back side of a user. By the actuator 14 winding the belt body 13 (first belt 16), tension acts on the first belt 16 and the second belt 17. The tension causes generation of an assist force for assisting the user's work, which reduces a burden on the body of the user.

For example, when a user (caregiver) changes his/her posture from a forward-tilted posture to an upright posture while holding a load (care-receiver) with his/her hands (see FIG. 18), tension acts on the belt body 13 by the actuator 14 winding the belt body 13. The tension makes it easy for the user to change his/her posture from a forward-tilted posture to an upright posture and thus reduces a burden on the body of the user. In other words, tension acting on the belt body 13 is generated by the actuator 14 as an assist force.

Also, the belt body 13 is non-stretchable over an entire length thereof. Consequently, a relationship between an output of the actuator 14 and tension acting on the belt body 13 becomes clear, which makes it easy to control a necessary assist force.

Upon the second harnesses 12 being attached to leg regions BL of the user and the user walking with the belt body 13 tensed, the leg regions BL may be pulled by the belt body 13. In the second belt 17, if the length of the left second belt portion 19 and the length of the right second belt portion 20 are always constant and fixed, the user may feel uncomfortable when the user walks.

Therefore, in the assist device 10 according to Embodiment 2, the one end portion 17a side of the second belt 17 is attached to the left second harness 12 and the other end portion 17d side of the second belt 17 is attached to the right second harness 12. Then, the second attachment portion 28 included in the joining member 18 supports the second belt 17 in such a manner that the second belt 17 is folded at an intermediate point and is movable in a longitudinal direction thereof.

With this configuration, when the user walks, in the second belt 17, the length of the left second belt portion 19 and the length of the right second belt portion 20 are changed. Therefore, the user can easily walk.

Furthermore, in Embodiment 2, as illustrated in FIG. 13, the second attachment portion 28 of the joining member 18 includes the rotary pulley 29 supported on a shaft portion 28a integrated with a first attachment portion 27. The second belt 17 is hung on the rotary pulley 29 so as to be folded at an intermediate point of the second belt 17. Therefore, when the user walks, the length of the left second belt portion 19 and the length of the right second belt portion 20 are easily changed, enabling the user to even more easily walk.

Also, the assist device 10 includes the cover 32 that covers the actuator 14 and the belt cover 50 that connects with the covers 32 and covers a part of the belt body 13. This configuration enables the cover 32 and the belt cover 50 to prevent contact between the actuator 14 and the part of the belt body 13 and a care-receiver when the assist device 10 and the care-receiver come into contact with each other. Consequently, even if the assist device 10 and the care-receiver come into contact with each other, operation of the assist device 10 can be prevented from being hindered, and thus, the assist device 10 can operate smoothly.

### Alterations

In the assist device 10 according to Embodiment 2, a part of the belt body 13 is covered by the tubular belt cover 50. However, in carrying out the present disclosure, the belt cover is not limited to this example, and as illustrated in FIGS. 19 and 20, a belt cover 53 including a contractible portion that expands and contracts in a bellows-like manner may be used, or as illustrated in FIGS. 21 and 22, a belt cover 54 including a contractible portion that expands and contracts in a telescopic manner may be used.

FIGS. 19 and 20 are diagrams each illustrating a belt cover 53 according to an alteration of Embodiment 2. FIG. 19 illustrates the belt cover 53 in an expanded state. FIG. 20 illustrates the belt cover 53 in a contracted state. The belt cover 53 includes an upper opening 531 at one end and a lower opening 532 at the other end and has a hollow structure inside. The one end side of the belt cover 53 connects with a cover 32 of a control box 30 and the upper opening 531 connects with an opening 32a of the cover 32.

In a state in which at least a first belt 16 is wound (FIG. 20), the belt cover 53 covers an entirety of a part of the first belt 16, the part being exposed from the cover 32, and is in contact with a joining member 18 on the other end side. A width of the lower opening 532 at the other end is smaller than a width of the joining member 18 but is larger than a width of the first belt 16. In other words, the first belt 16 is provided so as to be movable inside the belt cover 53 but the joining member 18 cannot pass through the lower opening 532. The belt cover 53 has a bellows-like shape in which a plurality of protrusion portions 533 and a plurality of recess portions 534 are provided alternately.

Where the belt cover 53 is not biased in a direction of contraction of the bellows-like shape, as illustrated in FIG. 19, the belt cover 53 expands with the other end in contact with the joining member 18 because of its own rigidity. Note that in FIG. 19, a length of a part of the first belt 16, the part being exposed from the cover 32, is shorter than a length of the belt cover 53 when the belt cover 53 is not biased. From this state, if the first belt 16 is further unwound and the length of the part of the first belt 16, the part being exposed from the cover 32, becomes longer than the length of the belt cover 53 when the belt cover 53 is not biased, the other end of the belt cover 53 and the joining member 18 are separated from each other.

Upon the belt body 13 (more specifically, the first belt 16) being wound by the actuator 14, as illustrated in FIG. 20, the joining member 18 pushes the other end of the belt cover 53 upward. Consequently, the belt cover 53 contracts. Also, upon the belt body 13 being unwound by the actuator 14, as illustrated in FIG. 19, the joining member 18 moves downward, and along with the movement of the joining member 18, the belt cover 53 expands because of its own rigidity while being kept in contact with the joining member 18. In the belt cover 53, the plurality of protrusion portions 533 and the plurality of recess portions 534 form the contractible portion.

An assist device 10 according to the alteration enables contact between a care-receiver and the belt body 13 to be prevented by the belt cover 53, and thus, the assist device 10 can operate smoothly. Also, the belt cover 53 covers the entirety of the part of the first belt 16, the part being exposed from the cover 32, and thus can more reliably prevent contact between the belt body 13, which operates by being wound by the actuator 14, and a care-receiver.

Here, in order to help the care-receiver, the user takes various postures in a state in which the user wears the assist device 10. If the belt body 13 is wound or unwound in a posture in which either a breast region BC and/or shoulder regions BS are titled leftward or rightward relative to a waist region BW, the right side or the left side of the belt body 13 extending through the inside of the belt cover 53 may come into contact with an inner wall of the belt cover 53. In the present alteration, the belt cover 53 has a configuration that contracts along with winding of the belt body 13 and expands along with unwinding of the belt body 13. Therefore, even if the belt body 13 that is being wound comes into contact with the inner wall of the belt cover 53, the inner wall of the belt cover 53 also contracts in a direction that is the same as a direction in which the belt body 13 is wound, and thus, the winding of the belt body 13 is not hindered. Likewise, during unwinding of the belt body 13, the belt cover 53 expands in a direction that is the same as a direction of the unwinding of the belt body 13, and the unwinding of the belt body 13 is not hindered. Therefore, the assist device 10 can operate more smoothly.

FIGS. 21 and 22 are diagrams each illustrating a belt cover 54 according to another alteration of Embodiment 2. FIG. 21 illustrates the belt cover 54 in an expanded state. FIG. 22 illustrates the belt cover 54 in a contracted state. The belt cover 54 includes an upper opening 541 at one end and a lower opening 542 at the other end and has a hollow structure inside. The one end side connects with a cover 32 of a control box 30 and the upper opening 541 connects with an opening 32a of the cover.

The belt cover 54 covers an entirety of a part of the first belt 16, the part being exposed from the cover 32, and the other end side of the belt cover 54 is joined to a joining member 18. A width of the lower opening 542 at the other end is smaller than a width of the joining member 18 but is larger than a width of the first belt 16. In other words, while the first belt 16 is provided so as to be movable inside the belt cover 54, the joining member 18 cannot pass through the lower opening 542. The belt cover 54 includes a plurality of frame portions 543, 544, 545, a connecting portion 546 that connects the frame portion 543 and the frame portion 544 and a connecting portion 547 that connects the frame portion 544 and the frame portion 545, and has a telescopic shape. The plurality of frame portions 543, 544, 545 are provided in the order mentioned, the order being a descending order of inner diameter.

Upon the belt body 13 (more specifically, the first belt 16) being wound by the actuator 14 and the joining member 18 pushing the other end of the belt cover 54 upward, as illustrated in FIG. 22, the belt cover 54 contracts with the connecting portions 546, 547 folded, the frame portion 545 received inside the frame portion 544 and the frame portion 544 received inside the frame portion 543. Also, upon the belt body 13 being unwound by the actuator 14, as illustrated in FIG. 21, the joining member 18 moves downward, along with the movement of the joining member 18, the other end of the belt cover 54 joined to the joining member 18 is pulled downward, and the belt cover 54 expands accordingly. In the belt cover 54, the plurality of frame portions 543, 544, 545 and the plurality of connecting portions 546, 547 form the contractible portion.

An assist device 10 according to the other alteration enables contact between a care-receiver and the belt body 13 to be prevented by the belt cover 54, and thus, the assist device 10 can operate smoothly. Also, the belt cover 54 covers the entirety of the part of the first belt 16, the part being exposed from the cover 32, and thus can more reliably prevent contact between the belt body 13, which operates by being wound by the actuator 14, and a care-receiver.

### Others

For prevention of pain in the lower back of a user, it is preferable that the second harnesses 12 be fitted to leg regions BL. This is because a load on a waist region BW can be reduced by the second harness 12 being fitted to leg regions BL.

In each form of assist device 10, the belt body 13 is light in weight and can conform to the body of a user even if the user changes his/her posture, and thus follows movement of the user. Therefore, an assist device 10 that provides good wear comfort can be provided.

Note that in the above disclosure, the control box 30 is provided on the rear side (back side) of a user in the first harness 11 but may be provided on the front side of a user. In this case, the belt body 13 is provided along the back of the user through shoulder regions BS of the user.

The embodiments disclosed herein are mere examples in every respect and are not limiting. The scope of the right for the present disclosure is not limited to the above embodiments but includes all changes that come within the meaning and range of equivalency of the claims.

What is clamed is:

1. An assist device comprising:
a first harness configured to be fitted to at least one of a shoulder region and a breast region of a user;
a second harness configured to be fitted to a leg region or a waist region of the user;
a belt body provided so as to extend to the first harness and to the second harness along a back side of the user;
an actuator provided in the first harness or the second harness and configured to wind and unwind a part of the belt body; and
a grip portion to be held by a person other than the user, the grip portion being attached to at least one of the first harness and the second harness.

2. The assist device according to claim 1, wherein:
the first harness includes a back body portion that faces a back of the user and a shoulder belt for fixing the first harness to the user, the shoulder belt connecting with the back body portion; and
the grip portion is attached to the back body portion.

3. The assist device according to claim 2, wherein the grip portion has a loop shape jointly with the back body portion by opposite end sides of the grip portion being connected respectively to an upper portion and a lower portion of each of a left portion and a right portion of the back body portion.

4. The assist device according to claim 2, wherein:
the actuator is mounted on a base attached to the back body portion.

5. An assist device comprising:
a first harness configured to be fitted to at least one of a shoulder region and a breast region of a user;
a second harness configured to be fitted to a leg region or a waist region of the user;
a belt body provided so as to extend to the first harness and to the second harness along a back side of the user;
an actuator provided in the first harness and configured to wind and unwind a part of the belt body;
a cover configured to cover the actuator; and
a belt cover connected to the cover and configured to cover at least a part of the belt body.

6. The assist device according to claim 5, wherein:
the belt cover has a tubular shape including an opening at each of one end and another end, the one end connects with the cover, and the opening at the other end is provided below a position corresponding to the breast region on the back side of the user; and
the belt body is connected from the first harness to the second harness via the opening at the other end.

7. The assist device according to claim 5, wherein the belt cover includes a contractible portion configured to contract along with winding of the belt body.

8. The assist device according to claim 5, wherein:
the belt body includes a first belt configured to be wound and unwound by the actuator, a second belt attached to the second harness and a joining member that joins the first belt and the second belt; and
the belt cover is configured to cover an entirety of a part of the first belt, the part being exposed from the cover.
